(19) United States
(12) Patent Application Publication
(10) Pub. No.: US 2022/0133830 A1
(43) Pub. Date: May 5, 2022

(54) TOPICAL COMPOSITION FOR CONTROL OF DEMODEX

(71) Applicant: BRUDER HEALTHCARE COMPANY, LLC, Alpharetta, GA (US)

(72) Inventors: Mark H. BRUDER, Alpharetta, GA (US); Rodney L. DOBSON, Greer, SC (US)

(73) Assignee: BRUDER HEALTHCARE COMPANY, LLC, Alpharetta, GA (US)

(21) Appl. No.: 17/453,239

(22) Filed: Nov. 2, 2021

Related U.S. Application Data
(60) Provisional application No. 63/108,908, filed on Nov. 3, 2020.

(51) Int. Cl. A61K 36/61; A61K 9/00; A61K 31/05; A61K 31/045; A61K 9/107; A61P 33/14

(52) U.S. Cl. CPC A61K 36/61; A61K 9/0014; A61P 33/14; A61K 31/045; A61K 9/107; A61K 31/05

(57) ABSTRACT

A topical composition for treating overgrowth of Demodex mites. The topical composition preferably includes one or more active ingredients, optionally encapsulated in lipids. In example embodiments, the topical composition comprises a first active ingredient between either tea tree oil or terpinen-4-ol, and a second active ingredient of cannabidiol oil.

### CROSS-REFERENCE TO RELATED APPLICATION

This application claims the benefit of U.S. Provisional Patent Application Ser. No. 63/108,908 filed Nov. 3, 2020, the entirety of which is hereby incorporated herein by reference for all purposes.

### TECHNICAL FIELD

The present invention relates generally to the field of topical preparations, and more particularly to a topical composition for a dermatological preparation comprising tea tree oil (TTO) and cannabidiol (CBD) encapsulated in lipids intended for application to a user's skin or hair, such as eyelids and/or eyelashes, to fight the growth of Demodex mites.

### BACKGROUND

Demodex folliculorum and Demodex brevis are two species of Demodex mites typically found on the skin of human and animal subjects. Demodex mites can be found on both symptomatic and asymptomatic individuals and typically pose no serious threat or conditions in low densities. However, infestation of Demodex in excess or high numbers have been implicated in numerous dermatological conditions or demodicosis, especially around the eyes of some individuals, including anterior and posterior blepharitis, dry eye syndrome, meibomian gland dysfunction (MGD), ocular rosacea, blepharoconjunctivitis, and blepharokeratitis, among others.

Conventionally, medicated ointments with active ingredients, such as benzyl benzoate, salicylic acid, selenium sulfide, and sulfur, have been used to combat infestations of Demodex mites and treat demodicosis. However, such ingredients can cause harsh irritation and discomfort if exposed to the eyes of the individual users due to their intrinsic toxicity. Other methods of therapy, such as warm compresses and antibiotic/steroid combinations, may help reduce discomfort for the affected individuals, but such methods do not eradicate the root cause of the demodicosis—the infestation of Demodex mites on the individual's skin.

Currently, a commonly used agent in treating for Demodex is tea tree oil, which comprises the active ingredient terpinen-4-ol (T4O)—a terpene with antimicrobial, antifungal, antiviral, antiseptic, and acaricidal properties. In one study, TTO was credited with stimulating Demodex to migrate from the cylindrical dandruff, wherein the Demodex are frequently trapped, to the individuals' skin. See Gao, Y. Y., Di Pascuale, M. A., Li, W., Baradaran-Rafii, A., Elizondo, A., Kuo, C. L., Raju, V. K., & Tseng, S. C. (2005), In Vitro and in Vivo Killing of Ocular Demodex by Tea Tree Oil, The British Journal of Ophthalmology, 89 (11), 1468-1473. The study further found that 10% concentration of TTO was effective in arousing the Demodex to migrate out to the skin for mechanical removal but ineffective in killing the Demodex. 50% concentration of TTO, on the other hand, was much more effective in killing the Demodex but it also generated variable degrees of irritation in some individuals. Most commercially available TTO products contain 1.5% concentration or less of TTO to prevent any burning sensation or discomfort to the users. Therefore, for treatments—such as for Demodex—where user compliance is critical to its overall effectiveness, minimizing discomfort to the patients or users requires significant consideration.

Accordingly, it can be seen that needs exist for improved agents and methods for treatment of Demodex. It is to the provision of treatments meeting these and other needs that the present invention is primarily directed.

### SUMMARY

In example embodiments, the present invention provides a topical composition for treatment of Demodex mites. The topical composition includes a first active ingredient and a second active ingredient encapsulated in an excipient. In example embodiments, the first active ingredient is either tea tree oil or terpinen-4-ol, an extract component of tea tree oil. The second active ingredient is cannabidiol. In example embodiments, the first and second active ingredients are further encapsulated in lipids.

Optionally, the topical composition may include a tea tree oil concentration between 1.5% and 75%, or a terpinen-4-ol concentration between 1% and 50%. The topical composition may also have a concentration of cannabidiol between 0.01% and 2%. In example embodiments, the topical composition has a cannabidiol concentration of 0.5%

In another aspect, the invention relates to a dermatological preparation for example for treating Demodex mites. The dermatological preparation includes a first concentration of tea tree oil and a second concentration of cannabidiol oil. The tea tree oil and cannabidiol oils are encapsulated in lipids.

In example embodiments, the concentration of tea tree oil in the dermatological preparation is between about 1.5% and 75%. In other example embodiments, the concentration of cannabidiol oil in the dermatological preparation is between about 1% and 50%. In still other example embodiments, the dermatological preparation has an encapsulation rate between about 25% and about 95%.

In still another aspect, the invention relates to a method of treating the overgrowth of Demodex mites on individuals. In example forms, the method includes producing a topical composition comprising at least one active ingredient encapsulated in an excipient. The at least one active ingredient is optionally encapsulated in at least one lipid, for example in nano-lipid particles. The method further includes applying the topical composition to a treatment area on an individual. Optionally, the at least one active ingredient is either tea tree oil or terpinen-4-ol. Optionally, the method may include producing a topical composition comprising two active ingredients, the first being tea tree oil or terpinen-4-ol and the second being cannabidiol oil.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of example embodiments are explanatory of example embodiments of the invention, and are not restrictive of the invention, as claimed.

### DETAILED DESCRIPTION OF EXAMPLE EMBODIMENTS

The present invention may be understood more readily by reference to the following detailed description of example embodiments taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

There are two types of Demodex mites commonly found on the skin of individuals—Demodex folliculorum and Demodex brevis. While Demodex mites can be found on any part of the human body, they are more prevalent in areas immediately surrounding the eyes, such as the eyelids and eyelashes. For example, Demodex folliculorum are commonly found in eyelash follicles and Demodex brevis are found burrowed deep in the sebaceous and meibomian glands where the Demodex mites feed on sebum, a complex mixture of lipids produced and secreted by the sebaceous glands, as their main food source. Typically, Demodex mites are found in low densities and pose no serious threat or adverse condition to individuals. However, Demodex in high densities have been implicated with causing a variety of dermatological conditions, including anterior and posterior blepharitis, dry eye syndrome, meibomian gland dysfunction (MGD), rosacea, blepharoconjunctivitis, and blepharokeratitis, among others.

The present invention relates generally to a composition for topical preparations, such as for example lotions, gels, foams, or liquid, for treating overgrowth of Demodex mites and demodicosis. Generally, the topical composition comprises tea tree oil (TTO) or terpinen-4-ol (T4O) as a primary active ingredient and cannabidiol (CBD) isolate as a secondary active ingredient. The TTO/T4O and CBD components are optionally encapsulated in nano-lipid particles.

According to example embodiments of the present invention, the primary active ingredient in the topical composition is T4O, a component extract from TTO known to be an effective agent for treating Demodex mites. The concentration of TTO or T4O is directly related to the effectiveness of the topical composition in treating for Demodex mites wherein, generally, a TTO concentration of about 25% or greater is needed for a reasonably effective treatment for Demodex, while a T4O concentration of about 5% could yield similar treatment results. However, high concentrations of TTO or T4O may also cause discomfort to the users, such as for example creating burning and/or stinging sensations where applied or variable degrees of irritation of the eyes. As a result, most TTO products contain 1.5% or less of TTO concentration to prevent such discomforts at the cost of diminishing the effectiveness of the treatment at such low concentrations. The topical composition of the present invention seeks to provide a balance between an effective acaricide and preventing or minimizing discomfort to its users. In example embodiments of the present invention, the topical composition comprises between about 1% and 50% concentration of T4O or between about 1.5% and about 75% of TTO concentration, or more preferably about 5% concentration of T4O or between about 10% and about 15% of TTO concentration.

According to example embodiments, the topical composition further comprises cannabidiol (CBD) isolate as the secondary active ingredient. CBD isolate, or CBD oil, provides a soothing and calming effect for irritated eyes. In example embodiments, the topical composition comprises between about 0.01% and about 2.0% concentration of CBD oil, or more preferably about 0.5% concentration of CBD oil.

According to example embodiments of the present invention, the primary and secondary active ingredients—TTO/T4O and CBD, respectively—are at least partially encapsulated or entrained in lipids, for example nano-lipid particles. In example embodiments, the encapsulation rate of T4O and CBD components in the topical composition is between about 25% and about 95%. However, generally, encapsulation rates above 50% may be preferred.

The encapsulation of the TTO/T4O and CBD agents provides a number of advantages and benefits for the topical composition. First, encapsulation of the active ingredients results in a reduction of eye irritation and TTO-related odor which some individuals may find offensive or unpleasant. Second, encapsulation of T4O in lipids may provide a more effective treatment for combating the overgrowth of Demodex mites wherein Demodex mites which feed on lipids as their main source of food ingest the lipid encapsulated TTO/T4O and thereby killing the mites. Third, T4O and CBD oils encapsulated in nano-lipid particles can provide more rapid tissue/gland penetration and bioavailability, improving the effectiveness in treating Demodex and the permeation and uptake of lipid encapsulated CBD to sooth irritated glands and tissues more quickly. For treatments reliant on user compliance, including treatments for Demodex, minimizing discomforts (such as eye irritation and unpleasant odors) and improving user relief can be significant factors in the overall effectiveness or results of such treatments. Fourth, encapsulation of the T4O and CBD agents reduces the rate of oxidation of the active ingredients and thereby enhances product stability and shelf-life of the topical composition, as compared to unencapsulated ingredients.

In example embodiments, the topical composition is utilized to produce a topical lotion for applying to eyelids and eyelashes to fight overgrowth of Demodex. In other example embodiments, the topical composition may be utilized in producing other forms of topical preparations or delivery means, such as for example foams, gels, liquids, creams, ointments, oils, or other similar means. Such various preparations and delivery means may be incorporated into various forms of applicators, such as for example brush or foam applicators, roll-on applicators, sprays, pump dispensers, or other similar means. In some example embodiments, the topical composition may include additional active ingredients and/or excipients. In still other example embodiments, the topical composition may include only one encapsulated active ingredient.

In example methods of use, the topical composition is prepared in the form of a dermatological lotion comprising at least some concentration of TTO or T4O and at least some concentration CBD oil, wherein the TTO, T4O and/or CBD oil are encapsulated in lipids, for example in nano-lipid particles. The dermatological lotion is dispensed onto a clean fingertip of the user and applied to the affected skin or treatment area, for example the user's eyelids or another part of the user's body affected by demodicosis. Alternatively, the topical lotion may be dispensed onto an applicator sponge, wipe, or other similar applicators for application to the treatment area. According to example methods of use, the topical lotion is applied to the treatment area once per day or more frequently if the overgrowth of Demodex is severe. In other example methods of use, the topical composition may be applied as a prophylactic to prevent recurrence of Meibomian Gland Dysfunction and/or other Demodex related issues. In yet another example method of use, the topical composition may be used with compresses, such as eye compresses, to further improve or enhance treatments of various dermatological and/or ocular conditions, such as for example MGD, blepharitis, and dry eye syndrome.

While the invention has been described with reference to example embodiments, it will be understood by those skilled in the art that a variety of modifications, additions and deletions are within the scope of the invention, as defined by the following claims.

What is claimed is:

1. A topical composition for treatment of Demodex mites, the topical composition comprising a first active ingredient and a second active ingredient wherein the first and second active ingredients are encapsulated in an excipient.

2. The topical composition of claim 1, wherein the first active ingredient is a tea tree oil.

3. The topical composition of claim 2, wherein the topical composition comprises between 1.5% and 75% concentration of the tea tree oil.

4. The topical composition of claim 1, wherein the first active ingredient is a terpinen-4-ol.

5. The topical composition of claim 4, wherein the topical composition comprises between 1% and 50% concentration of the terpinen-4-ol.

6. The topical composition of claim 4, wherein the topical composition comprises 5% concentration of the terpinen-4-ol.

7. The topical composition of claim 1, wherein the second active ingredient is a cannabidiol.

8. The topical composition of claim 7, wherein the topical composition comprises between 0.01% and 2% concentration of the cannabidiol.

9. The topical composition of claim 8, wherein the topical composition comprises 0.5% concentration of the cannabidiol.

10. The topical composition of claim 1, wherein the excipient comprises at least one lipid.

11. The topical composition of claim 1, wherein the topical composition is a topical lotion.

12. A dermatological preparation comprising a first concentration of tea tree oil and a second concentration of cannabidiol oil, wherein the tea tree and cannabidiol oils are encapsulated in lipids.

13. The dermatological preparation of claim 12, wherein the first concentration of tea tree oil is between 1.5% and 75%.

14. The dermatological preparation of claim 12, wherein the second concentration of cannabidiol oil is between 1% and 50%.

15. The dermatological preparation of claim 12, wherein the dermatological preparation comprises an encapsulation rate between 25% and 95%.

16. A method of treating Demodex mites on a user, the method of treatment comprising:
producing a topical composition comprising at least one active ingredient encapsulated in an excipient comprising at least one lipid, and
applying the topical composition to a treatment area on the user.

17. The method of treating Demodex mites of claim 16, wherein the at least one active ingredient is a tea tree oil or terpinen-4-ol.

18. The method of treating Demodex mites of claim 16, wherein the topical composition comprises a first active ingredient and a second active ingredient.

19. The method of treating Demodex mites of claim 18, wherein the first active ingredient is tea tree oil and the second active ingredient is cannabidiol oil.

20. The method of treating Demodex mites of claim 16, wherein the excipient is a plurality of lipids.
